(19) United States
(12) Patent Application Publication
(10) Pub. No.: US 2022/0133522 A1
(43) Pub. Date: May 5, 2022

(54) INTESTINAL WASTE MANAGEMENT SYSTEM

(71) Applicant: SECRETARY, DEPARTMENT OF BIOTECHNOLOGY, New Delhi (IN)

(72) Inventors: Pranav Chopra, New Delhi (IN); Neeraj Kumar, Noida (IN); Vijay Thakur, Jabalpur (IN)

(21) Appl. No.: 17/433,866

(22) PCT Filed: Feb. 27, 2020
(86) PCT No.: PCT/IN2020/050179

§ 371 Date: Aug. 25, 2021

(30) Foreign Application Priority Data
Feb. 27, 2019 (IN) 201911007754

(51) Int. Cl. A61F 5/44; A61F 5/443

(52) U.S. Cl. CPC A61F 5/4405; A61F 2005/4486; A61F 5/443

(57) ABSTRACT

Intestinal waste management system (102) comprising a device (104) having an anchoring mechanism (106) for anchoring the device (104) once the device (104) is inserted into a stoma. A continence mechanism (108) controls discharge of intestinal waste to outside the intestinal waste management system (102). The continence mechanism (108) comprising abase flange (110) to be placed at the stoma. The base flange 110 to be connected to a discharge channel (114) to form a discharge passage (116) to transport the intestinal waste from intestine to outside, in an open state of the continence mechanism (108). A continence membrane (118) having a first end connected to a distal end of the discharge channel (114). In closed state of the continence mechanism (108), the continence membrane (118) is to fold upon itself to create a constriction within the discharge passage (116) to stop the intestinal waste from flowing out.

### TECHNICAL FIELD

The present subject matter relates to a medical device and, in particular, to intestinal waste management for colostomates and ileostomates or those with fecal incontinence.

### BACKGROUND

Many people need to undergo surgeries to treat a variety of gastrointestinal conditions. Ostomy is a surgical procedure that reroutes the normal movement of intestinal wastes, such as stool out of the body. During ostomy surgery of the bowel, an opening is created in the abdomen. A portion of the bowel is brought to skin surface on the abdomen, such that the portion protrudes out from the opening. The protruding portion of the bowel is called a stoma. The stoma allows the intestinal waste to exit from the body. Examples of fecal incontinence and ostomy surgery include ileostomy surgery and colostomy surgery.

### BRIEF DESCRIPTION OF DRAWING

The detailed description is described with reference to the accompanying figures. In the figures, the left-most digit(s) of a reference number identifies the figure in which the reference number first appears. The same numbers are used throughout the drawings to reference like features and components.

FIGS. 1a, 1b, 1c, 1d, and 1e illustrate various views of an intestinal waste management system, in accordance with an embodiment of the present subject matter.

FIGS. 2a, 2b, and 2c illustrate operational stages of the intestinal waste management system, in accordance with an embodiment of the present subject matter.

FIGS. 3a, 3b, 3c, and 3d illustrate components of a continence mechanism of the intestinal waste management system, in accordance with an embodiment of the present subject matter.

FIG. 4a illustrates a flatus management system of the intestinal waste management system, in accordance with an embodiment of the present subject matter.

FIG. 4b illustrates a flatus management system of the intestinal waste management system, in accordance with another embodiment of the present subject matter.

FIG. 4c illustrates a flatus management system of the intestinal waste management system, in accordance with yet another embodiment of the present subject matter.

FIGS. 5a, 5b, and 5c illustrate a fluid valve of the intestinal waste management system, in accordance with an embodiment of the present subject matter.

FIGS. 6a, 6b, 6c, and 6d illustrate an intestinal waste management system, in accordance with another embodiment of the present subject matter.

FIGS. 7a and 7b illustrate the intestinal waste management system in different modes of operation, in accordance with another embodiment of the present subject matter.

FIG. 8a illustrates the intestinal waste management system, in accordance with yet another embodiment of the present subject matter.

FIG. 8b illustrates a cross section of a base flange and a front switch of the intestinal waste management system, in accordance with yet another embodiment of the present subject matter.

FIG. 8c illustrates a bottom view of the front switch, in accordance with yet another embodiment of the present subject matter.

FIG. 8d illustrates a bottom view of the base flange, snap fitted onto the front switch, in accordance with yet another embodiment of the present subject matter.

FIG. 8e illustrates a top view of the base flange, snap fitted onto the front switch, in accordance with yet another embodiment of the present subject matter.

FIG. 8f illustrates the base flange and an odour control component of the intestinal waste management system, in accordance with yet another embodiment of the present subject matter.

### DETAILED DESCRIPTION OF DRAWINGS

Ostomy is a surgical procedure performed to reroute the normal movement of intestinal wastes, such as stool or faeces out of the body. During ostomy surgery of the bowel of the patient, a stoma is created to divert the flow of the intestinal wastes. Once the stoma is created surgically, the patient loses control over excretion of the intestinal wastes from the body. Accordingly, intestinal waste collecting means, such as a removable bag, is typically attached around the stoma to constantly collect the intestinal wastes. The removable bag is emptied as required. However, the patient may face a number of complex lifestyle challenges. Further, the patient may experience discomfort and a substantial reduction in physical activities due to several problems associated with conventional removable bags, such as pouch leakage, offensive odour, and so on. Further, having the removable bag attached to the stoma may affect peristomal skin, i.e., the skin surrounding a stoma on the outside surface of the abdomen. For instance, the peristomal skin may get exposed to intestinal wastes and other intestinal contents, thus, leading to peristomal skin irritation.

One conventional technique discloses a stomal insert with an inflatable fixation element and a cover for covering the stoma and a cap for sealing the opening. The stomal insert includes a cap with means to accommodate a waste collecting bag. The cap is a disposable component which has to be replaced each time the intestinal waste is removed, thereby, increasing the per usage cost for the patient. Further, the stomal insert is incapable of providing continence if the cap is not present or a spare cap is unavailable.

Another conventional technique discloses a device for flow control and collection of human waste by positioning the device in either the stoma or rectum of the person. The device has a rigid member inserted into the body opening. The device further includes an expandable membrane, such as a balloon to secure the device in the body opening upon inflation. The inflatable balloon attached within the passageway allows selective passage of the intestinal waste. The inflatable balloon expands radially towards the center of the passageway upon inflation and is in sealing engagement with itself, thereby, closing the passageway. To allow the intestinal waste to pass, the inflatable balloon is deflated. However, an external means, such as a syringe or a pump is required for inflating or deflating the balloon to close or open the passageway for waste matter, respectively. Further, although such technique may provide continence to a certain extent, the patient alone may not be able to carry out the process of inflating and deflating the balloon each time the intestinal waste is to be expelled out of the body. Further, such technique does not provide any continence over the flatulence. As a result, the patient may experience abdominal discomfort and distress.

The present subject matter describes a medical device, also referred to as an intestinal waste management system that may be used to manage intestinal waste by patients who have undergone ostomy surgery. In an embodiment, the intestinal waste management system may be provided with a device that is insertable inside a stoma on a patient's abdomen and a waste collection bag attachable to the device. The device comprises an insertable port and a continence mechanism to control discharge of intestinal waste from the device to the collection bag. In another embodiment, the device may be partially insertable or non-insertable and may be on the stoma of the patient. Having the continence mechanism helps make the intestinal waste management system a self-contained device which may not require a separate tool to operate, thus enabling self-care.

In accordance to an embodiment of the present subject matter, the device maybe an insertable device and may include an anchoring mechanism for anchoring the device to the patient's stoma and a continence mechanism to control discharge of intestinal waste from an intestine of the patient to the waste collection bag. In one example, the device includes a soft, pliable base flange and a front switch to control the device between an open state and a closed state. The base flange may be connected to a discharge channel to form a discharge passage for transporting intestinal waste from the intestine to outside the intestinal waste management system, in the open state of the continence mechanism. The discharge passage acts as a transition passage for the intestinal waste, including stool and ensures that there is no contact between the intestinal waste and peristomal skin during evacuation.

In one implementation, the continence mechanism may include a soft elastomeric thin walled cylindrical sleeve, also referred to as a continence membrane. The continence membrane includes a first end and a second end such that the first end of the continence membrane is connected to a distal end of the discharge channel and the second end is connected the front switch. The combined assembly of the continence membrane, the base flange, and the front switch constitute a self-contained continence mechanism of the device. In the default state, i.e., open state, the discharge channel and the continence membrane together create a continuous discharge passage for unrestricted flow of waste matter from within the intestine to outside the body without coming in contact with the stoma or the peristomal skin. Upon rotating the front switch to the closed state, the continence membrane folds upon itself, thereby, creating a constriction and closing the flow path for the waste matter within the discharge passage. In one example, the front switch may be rotated in a clockwise direction to fold the continence membrane and reach the closed state of the continence mechanism. In another example, the front switch may be rotated in an anti-clockwise to reach the closed state of the continence mechanism. The constriction created due to the continence membrane prevents the intestinal waste from exiting the device. further, rotating the front switch in an opposite direction may unfold the membrane to again open the discharge passage to allow stool to exit the device, thus providing continence to the user.

The present subject matter, thus discloses a safe and an economical device for intestinal waste management. The device can be easily operated in a closed or an open state by simply rotating the front switch. As a result, the device does not require separate tool to operate, thus, enabling self-care. Further, the patient may not be required to attach a waste collection bag at all times. Instead, the patient may attach the waste collection bag only when the patient wishes to expel the intestinal waste. As a result, the user may not experience any discomfort that was earlier caused by the waste collection bag and would be able to carry out physical activities more efficiently. Accordingly, the user may now be able to empty the contents of the intestinal waste at desired timings. In one example, the device may be left in an inserted position for extended durations, for example, from 1 day to 45 days, thus improving the quality of life of the patient.

Further, the continence membrane in the closed state creates a constriction that restricts the intestinal waste from coming out, thereby preventing several problems, such as leakage, offensive odour, and contact with the peristomal skin, that would have been faced by the patient using the conventional devices. Further, in the open state, the continence membrane takes a cylindrical shape and is easily accessible for a user to clean the remnants of the waste matter left behind after evacuation. The user may simply wipe the surface of the membrane using a tissue or cloth or wet wipe to clean the continence membrane thus enhancing the usability of the device for self-care. In this way, the intestinal waste management system may provide a unique way of occluding the waste channel without compromising the safety of the user. Another advantage of the present subject matter over conventional techniques relates to the inherent safety of the intestinal waste management system. In emergent or urgent situations, the user may easily remove the intestinal waste management system from their stoma without the use of any external tool as required in conventional devices, for example, or deflation/deactivation of anchoring mechanisms.

Further, the continence mechanism of the intestinal waste management system provides a unique way of occluding the discharge channel without compromising the safety as no moving or twisting parts come in contact with human tissue. The intestinal waste management system further provides an easy to clean continence mechanism. In the open position, the continence membrane may take a cylindrical shape and may be easily accessible for a user to clean the remnants of the waste matter left behind after evacuation. The user may simply wipe the surface of the continence membrane using a tissue or cloth or wet wipe to clean the continence membrane thus enhancing the usability of the device for self-care. Further, in accordance to other embodiments of the present subject matter, the device maybe either a partially insertable or a non-insertable device. In said embodiment, the device may thus be used by people having a stoma with sensitive intestinal lining and may thus be uncomfortable using insertable or partially insertable anchoring mechanisms.

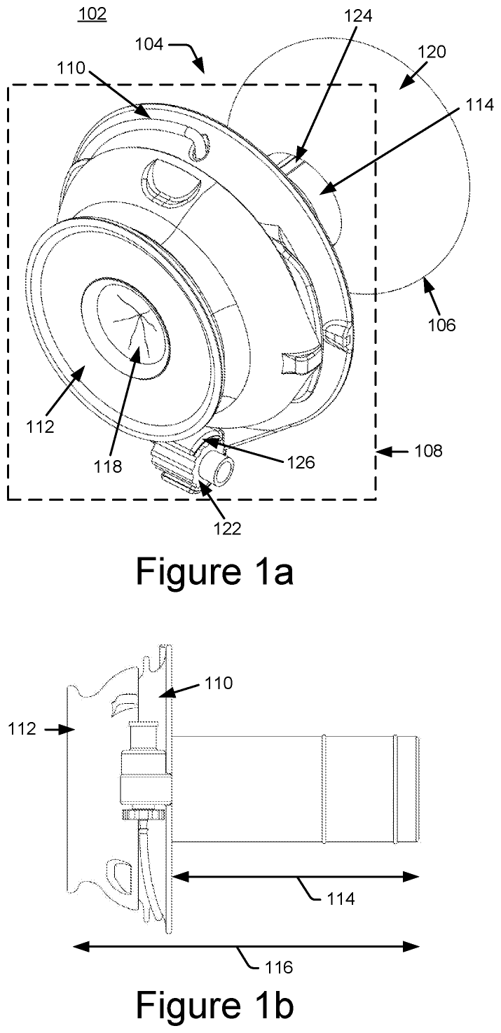

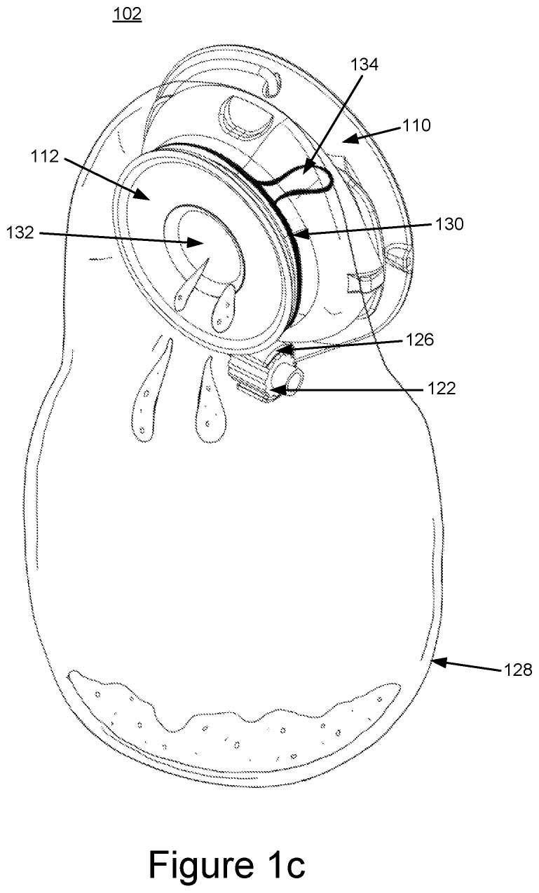

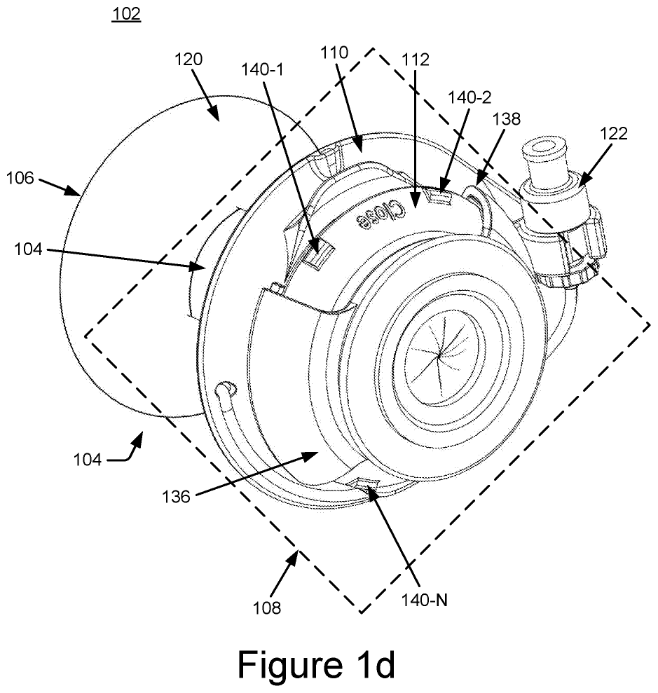

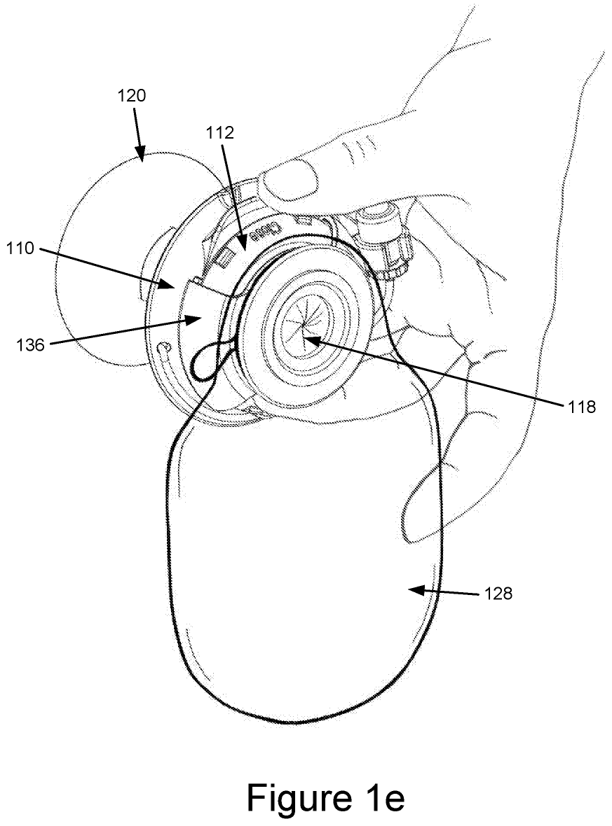

The present subject matter is further described with reference to FIGS. 1 to 8. It should be noted that the description and figures merely illustrate principles of the present subject matter. Various arrangements may be devised that, although not explicitly described or shown herein, encompass the principles of the present subject matter. Moreover, all statements herein reciting principles, aspects, and examples of the present subject matter, as well as specific examples thereof, are intended to encompass equivalents thereof.

FIG. 1a illustrates a perspective view of an intestinal waste management system 102, in accordance with an embodiment of the present subject matter. In one example, the intestinal waste management system 102 may be used by an ostomate, interchangeably referred to as a patient or a user, after an ostomy surgery. In one example, the intestinal waste management system 102, hereinafter interchangeably referred to as the system 102 includes a device 104 for excreting intestinal waste and a waste collection bag to receive the intestinal waste. In said embodiment, the device 104 is insertable inside a stoma on a patient's abdomen. FIG. 1b illustrates a side view of the intestinal waste management system 102, in accordance with an embodiment of the present subject matter. FIG. 1c illustrates a perspective view of the intestinal waste management system 102 coupled to a waste collection bag, in accordance with an embodiment of the present subject matter. FIG. 1d illustrates a perspective view of an intestinal waste management system 102, in accordance with another embodiment of present subject matter. FIG. 1e illustrates a perspective view of the intestinal waste management system 102 coupled to a waste collection bag, in accordance with another embodiment of the present subject matter.

Referring to FIGS. 1a and 1b, the device 104 includes an anchoring mechanism 106 for anchoring the device 104 within the stoma and a continence mechanism 108 to control discharge of intestinal waste from intestine of the patient to outside the intestinal waste management system 102. In one example, the device 104 may be anchored once the device 104 is inserted into the stoma of the patient.

In one example, the continence mechanism 108 includes a base flange 110 and a front switch 112 to control an operational state of the device 104 between an open and a closed state. The base flange 110 may be placed at an outer periphery of the stoma and may be connected to a discharge channel 114 to form a discharge passage 116 to transport intestinal waste from the intestine to outside the intestinal waste management system 102, in an open state of the continence mechanism 108. In one example, the discharge passage 116 may connect a proximal end of the base flange 110 to a distal end of the anchoring mechanism 106 to allow transport of intestinal waste from the intestine to outside the intestinal waste management system 102. The discharge passage 116 may thus act as a transition passage for the intestinal waste, including stool and ensures that there is no contact between the intestinal waste and peristomal skin during evacuation.

In one example, the length of the discharge channel 114 may be customizable for users depending on various parameters, such as abdominal wall thickness of the user. As each individual may have different abdominal wall thickness depending on the size of the fat layer, therefore, to accommodate users with different sizes the intestinal waste management system 102 may be provided as small, medium, large, etc. with different lengths of the discharge channel 114 to accommodate ostomates with different abdominal wall thickness. In one example, the length of the discharge channel 114 may vary in a range between 30 millimeter (mm) to 100 mm.

Further, the continence mechanism 108 includes a continence membrane 118 having a first end connected to a distal end of the discharge channel 114 and a second end connected to a proximal end on an inner surface of the front switch 112. In one example implementation, the continence membrane 118 may be a soft elastomeric thin walled cylindrical sleeve. In a default state, i.e., an open state of the continence mechanism 108, the continence membrane 118 may be in an open state such that the discharge passage 116 and the continence membrane 118 may together create a continuous channel for unrestricted flow of waste matter from within the intestine to outside of the body without coming in contact with the stoma or the peristomal skin.

To operate the continence mechanism 108 in a closed state, the front switch 112 may be rotated in a first direction to bring the continence membrane 118 in a closed state. In the closed state, the continence membrane 118 may fold upon itself to create a constriction within the discharge passage 116 to stop the intestinal waste from flowing out from the intestinal waste management system 102. The constriction created due to the continence membrane 118 prevents the intestinal waste from exiting the device 104 in a closed state of the continence mechanism 108. Rotating the front switch 112 in a second direction, opposite to the first direction, may unfold the continence membrane 118 and again opens the discharge passage 116 to allow stool to exit the device 104, thus providing continence to the user. In one example, the first direction may be a clockwise direction and the second direction may be an anti-clockwise direction. In another example, the first direction may be the anti-clockwise direction and the second direction may be the clockwise direction.

In one example, the anchoring mechanism 106 includes a fixation element 120 disposed at a proximal end of the device 104 to anchor the intestinal waste management system 102 inside the intestine once the device 104 of the intestinal waste management system 102 is inserted into the intestine. In one implementation, the fixation element 120 may be an inflatable cuff or balloon. The fixation element 120 may be inflated with an inflation medium to provide anchor against the inner wall of the peritoneal cavity without obstructing the discharge passage 116. In one example, the length of the discharge channel 114 may be such that the fixation element 120 is located just within the peritoneal cavity of the user. The fixation element 120 thus acts as an anchor against the intestinal waste management system 102 and helps preventing the device 104 from being pushed out by peristalsis motion. Anchoring the device 104 thus prevents the intestinal waste management system 102 from being dislodged while in use. Further, to remove the intestinal waste management system 102, the fixation element 120 may be deflated.

Further, the pressure exerted by the fixation element 120 on the intestinal lining may be predetermined to be between 0 psi to 5 psi to prevent any injury to the intestinal tissue. In one example, the fixation element 120 may be made from an elastomer material (preferably silicone rubber) with shore hardness between Shore 5A to 40A and a wall thickness between 0.1 mm to 0.6 mm. In one implementation, the fixation element 120 may be pre-shaped as a toroid to receive a fixed volume of fluid. Alternatively, the fixation element 120 may be entirely elastic and may be inflated to take a toroidal shape.

In one example, the inflation medium of the fixation element 120 may be a gaseous or a liquid material. In one embodiment, the fixation element 120 may be inflated with a measured quantity of water or air (20 mL-60 mL) pumped through an inflation device, such as a syringe. The device 104 further includes a fluid valve 122 to regulate inflow and outflow of the inflation medium from the fixation element 120 to control inflation and deflation of the fixation element 120. In one example, the fluid valve 122 may be a standard one-way check valve attached to an inflation tubing 124 of the anchoring mechanism 106 by means of a luer connector. To deflate the fixation element 120, the user may simply disconnect the fluid valve 122 from the luer fitting to allow the inflation medium to be pushed out by the elastic action of the fixation element 120. Alternatively, a luer activated one-way check valve may be used in case of preformed fixation element 120. In said example, a luer lock connection syringe may be used to release the inflation medium from the fixation element 120. In one example, the anchoring mechanism 106 may further include a groove (not shown in this figure) for the inflation tubing 124.

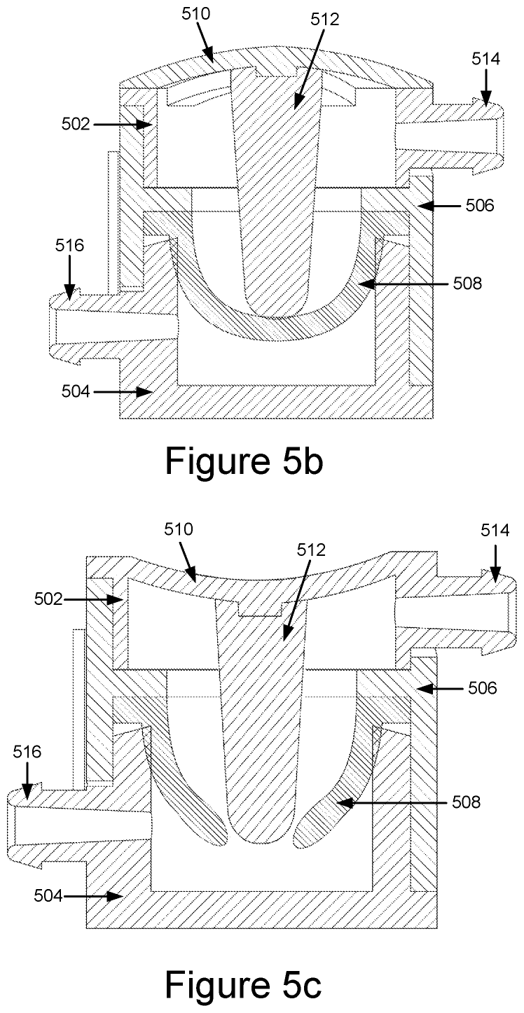

In another example, a specially designed fluid valve 122 that acts as a regular one-way check valve during inflation, may be used. To deflate the fixation element 120, a button on the fluid valve 122 may be pressed to over-ride the one-way valve and allow the fixation element 120 to deflate. In one example, the fluid valve 122 and the inflatable fixation element 120 may be connected by means of standard tubing with Inner Diameter (ID) typically 0.5 mm to 2 mm. In another example, the fluid valve 122 and the fixation element 120 may be connected by means of a single tube or multitude of tubes to increase the flow area and to provide redundancy in case one tube gets pinched or kinked during use. Alternatively, the fluid paths may be moulded within the discharge channel 114. In one example, the continence membrane 118 may include a fluid valve grip 126 to hold the fluid valve 122. The fluid valve 122 is further described with reference to FIGS. 5a-5c.

The intestinal waste management system 102 further includes a waste collection bag 128, as illustrated in FIG. 1c. As illustrated, the waste collection bag 128 may be attached to an outer ring feature of the front switch 112 to receive the intestinal waste during evacuation of the intestinal contents. In an embodiment, the waste collection bag 128 may be designed with an opening 130 on the top end of a flat face to enable attachment of the waste collection bag 128 to the front switch 112, in a vertical orientation as shown in FIG. 1c.

In one example, the mouth or opening 130 of the waste collection bag 128 may have a securing arrangement, such as a string 134 for securing the waste collection bag 128 to the device 104. The user may attach the waste collection bag 128 to the device 104 by mounting the opening 130 of the waste collection bag 128 over the front switch 112 such that the waste collection bag 128 completely encapsulates a discharge opening 132 of the continence mechanism 108. Once the waste collection bag 128 is attached to the device 104, the user may pull on the string attachment to secure the opening 130 of the waste collection bag 128 around the device 104.

In another example, the opening 130 of the waste collection bag 128 may have an elastic ring embedded in the opening 130 to ensure that the opening 130 snugly closes around the ring on the front switch 112. Alternatively, the opening 130 of the waste collection bag 128 may include a combination of elastic and string arrangement for additional securement.

Once the waste collection bag 128 is secured to the device 104, the user may move the front switch 112 to an open state. The intestinal contents may then start coming out of the discharge opening 132 on the front switch 112 and fall straight into the waste collection bag 128. After the contents of the intestine are emptied, the user may move the front switch 112 to the closed state, thereby closing off the stool passage pathway. The user may subsequently remove the waste collection bag 128 by loosening the string arrangement and detaching the waste collection bag 128 from the device 104. The waste collection bag 128 can then be disposed of in a safe manner. Alternatively, the user may wash the waste collection bag 128 after emptying the contents of the waste collection bag 128 in the toilet for reuse.

In an alternate embodiment, a drainage tube may be connected to the front switch 112 to allow direct drainage of the intestinal waste into the toilet. The drainage tube may be connected to the front switch 112 by means of one or more mechanical connections like bayonet lock, luer taper connection or any other technique known in the art. In one example, the user may connect the drainage tube to the front switch 112 as per their requirement for emptying their bowel contents. The length of the drainage tube may be such that it allows the user to defecate while standing as well as while sitting on the toilet seat. Such embodiment may be more useful for ileostomates and the users whose intestinal waste has more liquid consistency.

FIG. 1d illustrates the intestinal waste management system 102, in accordance with another embodiment of the present subject matter. In said embodiment, the device 104 includes the front switch 112, a switch housing 136, and the base flange 110. The switch housing 136 may be disposed on the base flange 110 and the front switch 112 to restrain an axial movement of the front switch 112. The switch housing 136 may restrain any axial movement of the front switch 112 while the front switch 112 is free to rotate about a central axis independent of the switch housing 136 and the base flange 110.

In one implementation, the switch housing 136 may include at least one cut-out window 138 on the outer circumference to allow physical access to one or more hand grips 140-1, 140-2, . . . , 140-N provided on the front switch 112 to operate the front switch 112. The one or more hand grips 140-1, 140-2, . . . , 140-N are hereinafter collectively referred to as hand grip 140 and individually referred to as hand grip 140. The switch housing 136 is attached with the base flange 110 to coalesce into a single body. In one example, the switch housing 136 may be joined to the base flange 110 by means of gluing or mechanical fixation or any other joining means known in the art.

FIG. 1e illustrates a waste collection bag 128 attached to the device 104 of the intestinal waste management system 102 having the switch housing 136, in accordance with another embodiment of the present subject matter. As illustrated in FIG. 1e, the waste collection bag 128 may be mounted on a non-rotating member element, i.e., the switch housing 136 of the insertion device 104. The user may thus open or close the continence mechanism 108 without the issue of the waste collection bag 128 rotating along with the motion of the front switch 112. In one example, the waste collection bag 128 may be connected to an outer ring feature of the switch housing 136 to enable capturing of the intestinal waste.

In one example, the intestinal waste management system 102 may also include an intestinal pressure measurement and monitoring unit. The intestinal pressure measurement and monitoring unit may include a sensor placed either within the discharge channel 114 or integrated within the continence membrane 118. The sensor may constantly measure the intestinal pressures developing with intestine of the patient. The intestinal pressure measurement and monitoring unit may further include a communication module to communicate pressure information indicating the intestinal pressure to a user device. in one example, the user device maybe a dedicated device or a user's personal device, such as a mobile phone. Further, the communication module may communicate with user device using a communication channel, for example, as Bluetooth, General Packet Radio Service (GPRS), WiFi etc. The pressure information may be used by the user device to monitor and guide user on safety as well as regular bowel movements. For example, once the intestinal pressures exceed a certain threshold, say, 150 mm of water for a sufficiently continuous period of time, for example, 20 mins, the communication module may send an alarm or message to the user as a reminder to go to the toilet for bowel evacuation. The communication module may alarm may also be used for safety to alert the user if the pressure exceeds safety level. For example, the communication module may send an alarm to the user device if the intestinal pressure exceeds a predetermined threshold level, for example 350 mm of water, to alert the user to remove the device 104 and seek help.

The intestinal pressure measurement and monitoring unit may also include an inflation pressure monitor to monitor if the user has filled the fixation element 120 upto a predetermined level, while also limiting the user from over-inflation. The pressure monitor or indicator may be in the form of a pilot balloon to indicate appropriate fill pressure or an electronic sensor or any other pressure measurement means known in the art.

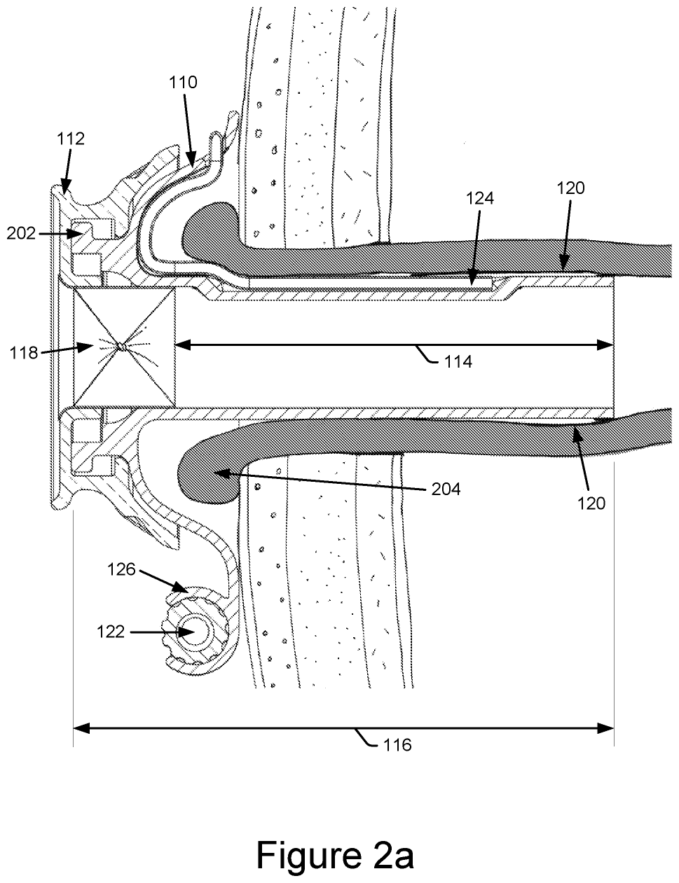

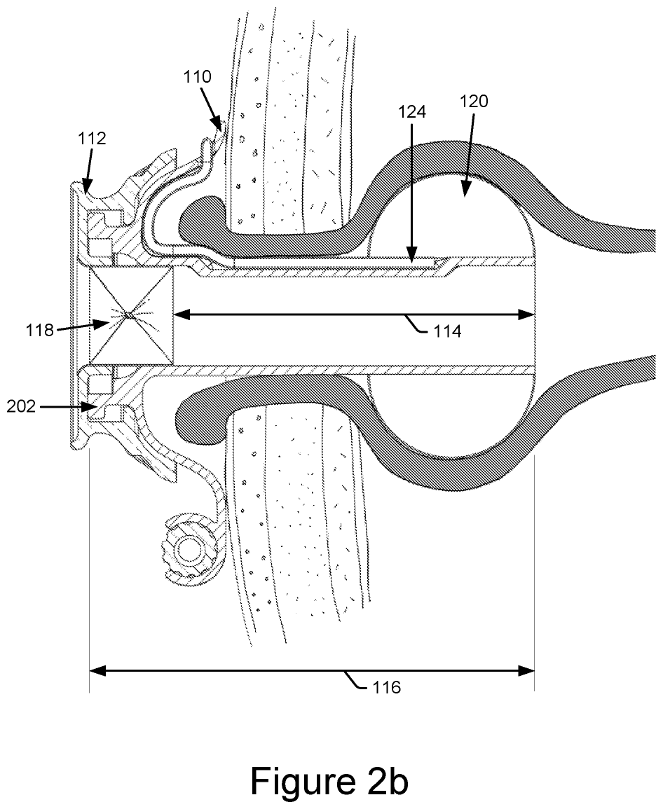

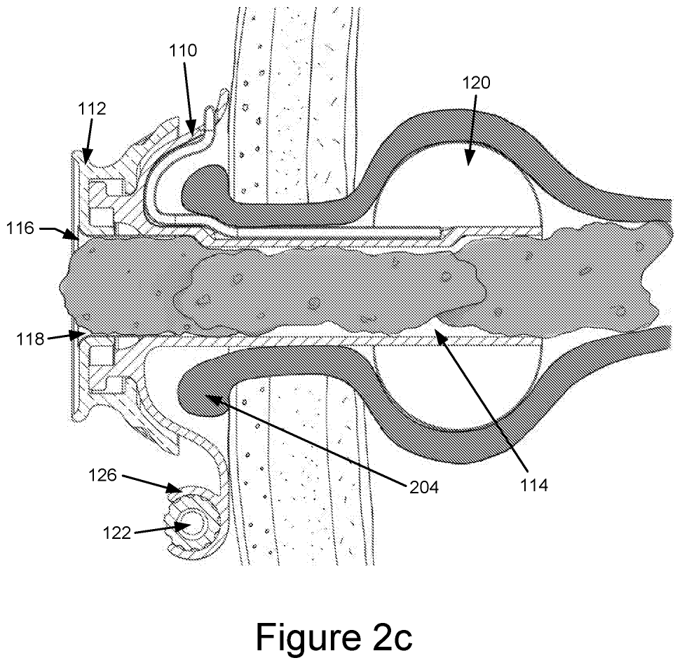

FIGS. 2a, 2b, and 2c illustrate operational stages of the continence mechanism 108 of the intestinal waste management system 102, in accordance with an embodiment of the present subject matter. FIG. 2a illustrates the continence mechanism 108 with the continence membrane 118 in the closed state and the fixation element 120 in the deflated state. FIG. 2b illustrates the continence mechanism 108 with the continence membrane 118 in the closed state and the fixation element 120 in the inflated state. FIG. 2c illustrates the continence mechanism 108 with the continence membrane in the open state, allowing the stool to exit the device 104.

As illustrated in FIG. 2a, the front switch 112 may be snap fit into a retention ring 202 of the base flange 110 to assemble the front switch 112 in the device 104. Snap fitting the front switch 112 into the retention ring 202 on the base flange 110 facilitates in restricting axial movement of the front switch 112 relative to the base flange 110. The front switch 112 however is free to rotate about the central axis of the device 104.

In operation, to use the intestinal waste management system 102, the user may insert the device 104 into the stoma on a patient's abdomen. In one example, the device 104 of the intestinal waste management system 102 may be inserted into the stoma using an applicator. In another example, the intestinal waste management system 102 may be inserted into the stoma directly without using an external device. Upon insertion of the device 104, the fixation element 120 of the anchoring mechanism 106 may be inflated using an inflation device to act as an anchor against the inner wall of the peritoneal cavity, as illustrated in FIG. 2b. The fixation element 120 is located at the proximal end of the device 104 and remains inside the stoma 204 and prevents the device 104 from being pushed out by peristalsis motion of the intestine. In one implementation, the fixation element 120 may be inflated to take a shape of toroid, thereby providing anchoring without obstructing the discharge passage 116. In the said implementation, the fixation element 120 may be inflated with a measured quantity of water or air (20 mL-60 mL) pumped through the inflation device, such as a syringe.

Once the device 104 is anchored, the user may continue with their daily activities with the continence mechanism 108 in the closed state as illustrated in FIG. 2b. As previously described, in the closed state, the continence membrane 118 folds upon itself to create a constriction within the discharge passage 116, thereby, closing the flow path for the waste matter within the discharge passage 116. The constriction created due to the continence membrane 118 prevents the intestinal waste from exiting the device 104.

Subsequently, whenever the user of the device 104 desires to empty the contents of the intestine, the user may attach the waste collection bag 128 to an outer ring feature of the front switch 112 to enable capturing of the intestinal waste during evacuation of the intestinal content, as previously illustrated in FIG. 1c. Once the waste collection bag 128 is secured to the device 104, the user may rotate the front switch 112 to move the continence mechanism 108 into the open state.

In one example, the user may operate the front switch 112 in the second direction to open the continence membrane 118 for operating in the open state of the continence mechanism 108. As previously described, in the open state, the discharge passage 116 and the continence membrane 118 together create a continuous channel for unrestricted flow of waste matter from within the intestine to outside of the body without coming in contact with the stoma or the peristomal skin. The intestinal contents may then start coming out of the discharge passage 116, as illustrated in FIG. 2c. The intestinal waste may subsequently fall straight into the waste collection bag 128. After the contents of the intestine are emptied, the user may move the front switch 112 to the closed state, thereby closing off the stool passage pathway. The user may further remove the waste collection bag 128 from the device 104. The user may empty the contents of the waste collection bag 128 and then dispose of the waste collection bag 128 in a safe manner. Alternatively, the user may wash the waste collection bag 128 after emptying the contents of the waste collection bag 128 for reuse.

In an alternate embodiment, a drainage tube may be connected to the front switch 112 to allow direct drainage of the intestinal waste into the toilet as described previously.

Subsequently, if the user wishes to remove the device 104 from the stoma 204, they may deflate the fixation element 120 using the fluid valve 122. The user may disconnect the fluid valve 122 from the leur fitting such that the inflation medium is pushed out by the elastic action of the fixation element 120. Alternatively, luer activated one-way check valve may be used in case of preformed fixation element 120. In said example, a luer lock connection syringe may be used to release the inflation medium from the fixation element 120.

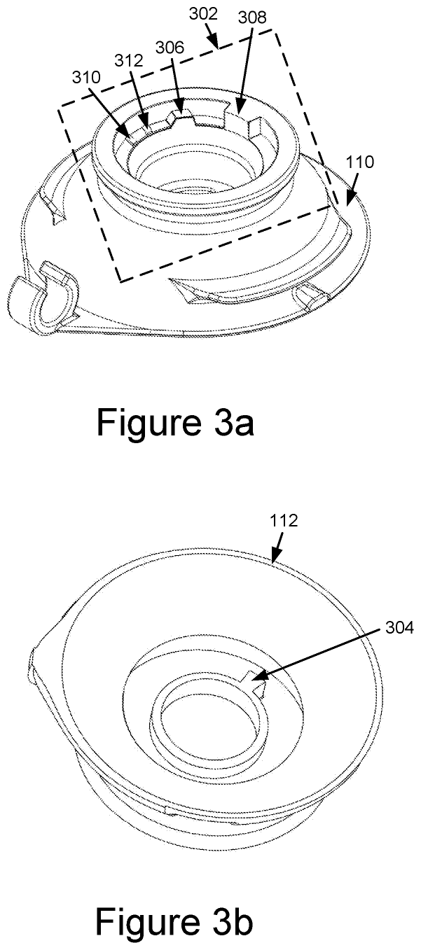

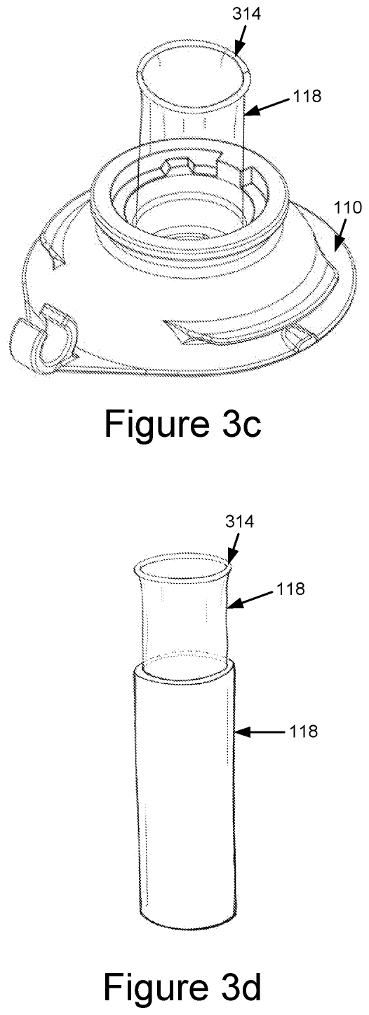

FIGS. 3a, 3b, 3c, and 3d illustrate components of a continence mechanism of the intestinal waste management system, in accordance with an embodiment of the present subject matter. FIG. 3a illustrates a top view of the base flange 110 illustrating a rotation control mechanism 302 of the continence mechanism 108. FIG. 3b illustrates a bottom view of the front switch 112 having a rotation control lug 304 of the continence mechanism 108. FIG. 3b illustrates a bottom view of the front switch 112 having a rotation control lug 304 of the continence mechanism 108. FIG. 3c illustrates a top view of the base flange 110 illustrating the continence membrane 118 of the continence mechanism 108. FIG. 3d illustrates the continence membrane 118 of the continence mechanism 108 and the discharge channel 114.

In one example implementation, the continence mechanism 108 further includes the rotation control mechanism 302 provided on the base flange 110 and the rotation control lug 304 protruding from the front switch 112 to control movement of the front switch 112 for opening or closing the continence membrane 118. As illustrated in FIGS. 3a and 3b, the rotation control lug 304 is provided on an inner surface of the front switch 112, protruding from the front switch 112. In one example, the rotation control lug 304 is to engage with the rotation control mechanism 302 to control a rotational movement of the front switch 112 along the outer surface of the base flange 110.

In one example implementation, the rotation control mechanism 302 may include a control step 306, an end stop 308, a first step 310, and a second step 312 as illustrated in FIG. 3a. In another example, the rotation control mechanism 302 may include the control step 306 and the an end stop 308, as illustrated in FIG. 3c. The end stop 308 is to engage with the rotation control lug 304 to stop the front switch 112 from rotating in a first direction in the closed state of the continence mechanism 108 and in a second direction in the open state of the continence mechanism 108. For instance, when the user is rotating the front switch 112 in the first direction, say, the clockwise direction, the rotation control lug 304 may strike and engage with the end stop 308 as the front switch 112 completes one full rotation for closing the continence membrane 118. The end stop 308 may thus provide a feedback to the user that the continence mechanism 108 is in a ‘closed’ position and prevent the front switch 112 from moving more than one full rotation. The rotational control mechanism 302 thus allows the front switch 112 to rotate freely in the clockwise direction for a fixed angular rotation.

In one example, the front switch 112 may require a total rotation angle in the range of 150 deg-300 deg to ‘close’ the continence mechanism 108. The angle to ‘close’ may depend on various parameters, such as free assembled length of the continence membrane 118 and free assembled diameter of the continence membrane 118. In one example, the assembled free length of the continence membrane 118 may vary in the range of 4-15 mm. In one example, the assembled diameter of the continence membrane 118 may be in the range of 10-35 mm. Similarly, the end stop 308 may engage with the rotation control lug 304 when the front switch 112 is being rotated in the second direction in the open state of the continence mechanism 108 to prevent further movement of the front switch 112.

Further, the first step 310 and the second step 312 may provide slight resistance to movement of the rotation control lug 304 to provide a haptic feedback to the user as an indication that the continence mechanism is about to ‘close’. Further, in the ‘close’ state of continence mechanism 108, as the front switch 112 is rotated in the second direction to unfold the continence membrane 118, the rotation control lug 304 may engage with the control step 306 to prevent any unwanted opening of the continence membrane 118. As illustrated, the control step 306 may be provided alongside the end stop 308 such that if the front switch 112 accidently moves in the second direction from the closed state of the continence membrane 118, the rotation control lug 304 may strike against the control step 306 to restrict the movement of the front switch 112. In one example, the control step 306 may be of a smaller height than the end stop 308 so that the rotation control lug 304 may slide over the control step 306 when an appropriate force is applied to move the front switch 112 by the user, thus providing a detent force for the movement of the front switch 112 for rotation in the second direction.

As the user rotates the front switch 112 to open the continence membrane 118, from the ‘close’ state of the continence mechanism 108, the rotation control lug 304 may slide over a flat portion of the rotation control mechanism 302 on the base flange 110, thus introducing friction torque that may oppose any relative motion between the front switch 112 and the base flange 110. In one example, the friction is carefully controlled between a lower limit and an upper limit. The upper limit may be chosen to allow the user to overcome the friction and rotate the front switch 112 to open and close the continence mechanism 108. The continence membrane 118, when folded upon itself, has a tendency to unfold on its own due to its elastomeric nature. The lower limit of friction is selected to be large enough to prevent the continence membrane 118 from un-folding on its own. A twisting torque and untwisting torque for closing and opening the front switch 112, respectively, is shown in equations 1 and 2 below, respectively:

Twisting Torque(for closing)=Friction torque (1)

Untwisting Torque(for opening)=Friction Torque+Detent (2)

The twisting of the continence membrane 118 while closing the continence mechanism 108 may cause the continence membrane 118 to stretch and in turn pull the front switch 112 towards the base flange 110. This may lead to an increased normal reaction between the contacting surfaces of the front switch 112 and the base flange 110. The increased friction torque acting upon the front switch 112 is shown in equation 3 below, respectively:

Friction torque=Coeff of Friction*Normal Reaction*Radius of contact line (3)

In one example, the normal reaction is controlled by a pre-stretch introduced in the continence membrane 118 during assembly in the intestinal waste management system 102. The pre-stretch can be between 0% and 20% of the free length of the continence membrane 118. The torque required to open the intestinal waste management system 102 could be between 10 N-mm to 80 N-mm.

Further, the friction coefficient is dependent on the material of the front switch 112 and the base flange 110. In one example, the base flange 110 and the front switch 112 may be made of silicone elastomer. The friction between the base flange 110 and the front switch 112 when made from silicone elastomer can be very high and may be reduced by applying lubricious coating on either of the contacting surfaces. Alternatively, silicone grease may be applied between contacting surfaces of the base flange 110 and the front switch 112 during assembly. Alternatively, the front switch 112 may be made from material such as thermoplastic elastomers (TPE), thermoplastic polyurethane (TPU), and like, to reduce the coefficient of friction.

Further, as previously described, the continence membrane 118 is a soft elastomeric thin walled cylindrical sleeve, as illustrated in FIG. 3c. In one example, the second end of the continence membrane 118 includes a relatively harder ring like feature referred to as a continence membrane ring 314. The diameter of the continence membrane ring 314 is marginally bigger than the diameter of the discharge opening 132 of the front switch 112. While assembling the device 104, the continence membrane ring 314 may be squeezed and passed through the discharge opening 132 of the front switch 112 and released while the first end is connected with the distal end of the discharge channel 114 of the base flange 110. Once released, the continence membrane ring 314 comes back to its original state and firmly sits in groove of the front switch 112 owing to the pull from the second end fixed in the front switch 112. The continence membrane ring 314 thus keeps the continence membrane 118 in place without requiring any further process to attach it to the front switch 112.

Further, as illustrated in FIG. 3d, the continence membrane 118 may be pre-molded with the discharge channel 114 such that the first end of the continence membrane 118 is connected to the distal end of the discharge channel 114 while the second end includes the continence membrane ring 314

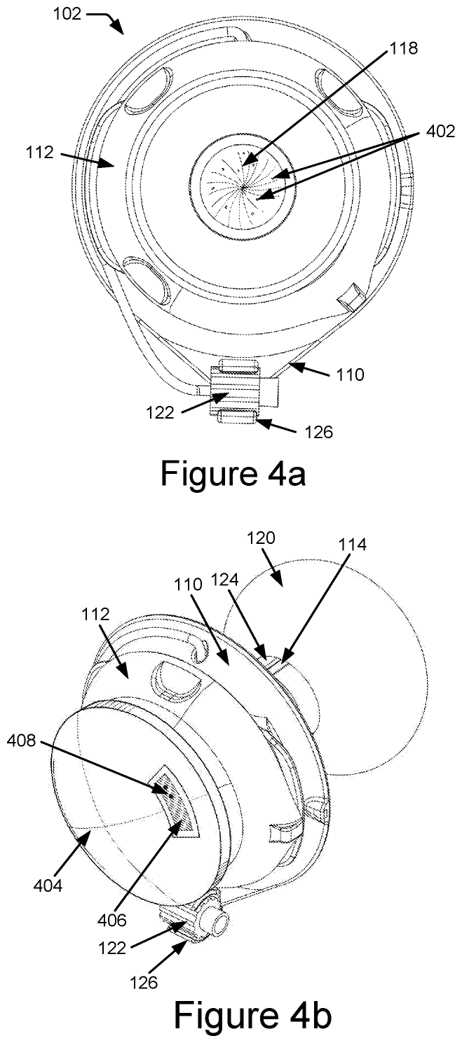

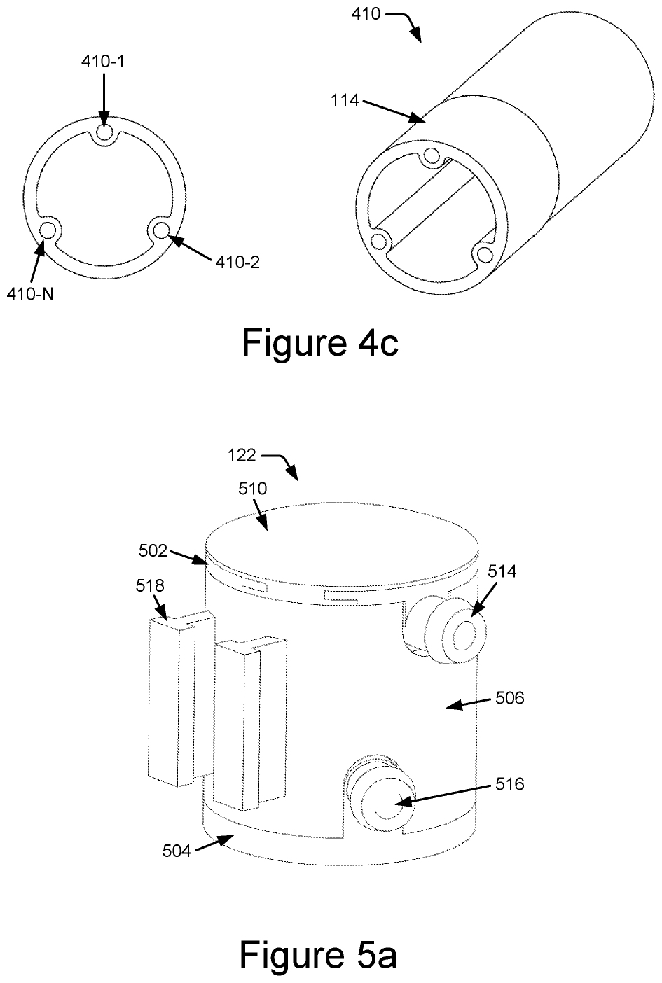

FIGS. 4a, 4b, and 4c illustrate various embodiments of a flatus management system of the intestinal waste management system 102, in accordance with an embodiment of the present subject matter. The flatus release mechanism may allow a controlled discharge of gaseous waste matter from the intestine to outside the intestinal waste management system 102. In one example, the various embodiments can be combined in a single embodiment such that the intestinal waste management system 102 may include a flatus management system having all the features described in FIGS. 4a, 4b, and 4c. In another example, various combinations of the features of the different embodiments can be combined in a single embodiment such that the intestinal waste management system 102 may include a flatus management system having one or more of the features described in FIGS. 4a, 4b, and 4c.

In one embodiment, the continence membrane 118 may function as the flatus management system to selectively allow gaseous waste matter to be released while not allowing the solid and liquid intestinal waste to pass through. To enable the continence membrane 118 to function as the flatus management system, the continence membrane 118 may be manufactured using microporous materials (example Expanded Polytetrafluoroethylene (PTFE). In this case, the continence mechanism 108 in its ‘closed’ position may completely block the flow path of the solid and liquid intestinal waste, while the gaseous waste (or flatus) on the other hand may safely pass through the microporous material.

In one example, the front switch 112 may be rotated to open the continence membrane 118 partially to selectively allow only gas to be released. In one example, the front switch 112 may be rotated up to the first step 310 provided on the base flange 110 to partially open the continence membrane 118 to a first partial position to allow the gaseous waste matter to escape from the intestine to outside the intestinal waste management system 102. The first step 310 in such a case may receive the rotation control lug 304 of the continence mechanism 108 and stop the rotation control lug 304 from moving forward. The first step 310 may thus act as an indication for the user operating the front switch 112 about the first partial position of the continence membrane 118.

In one example, the first step 310 may be positioned to provide an opening of a small angle. By rotating the front switch 112 by a small angle, the continence mechanism 108 may slightly unfold the continence membrane 118. Thereby, opening the constriction to create a very small opening (between 0.1 mm-0.5 mm diameter). Such a small opening may allow the gaseous contents of the intestine to escape while preventing any solid/semi-solid/or liquid contents from escaping. The range of angular rotation required to selectively open the continence mechanism 108 may be identified as the “gas release zone” between the “open” & “close” positions. The gas release zone may be between 100-250 degree of angular rotation.

Further, to allow more amount of flatus release, the front switch 112 may be rotated up to the second step 312 provided on the base flange 110 to partially open the continence membrane 118 to a second partial position. The second step 312 may receive the rotation control lug 304 for partially opening the continence membrane 118 up to the second partial position to allow the gaseous waste matter to escape from the intestine to outside the intestinal waste management system 102.

Further, the continence membrane 118 may include micro-perforations 402, as shown in FIG. 4a, to allow gaseous waste matter to escape in a closed state of the continence mechanism 108, while blocking the solid and liquid intestinal waste. In said example, the continence membrane 118 may be manufactured using standard elastomer materials, such as TPE, TPU or silicone elastomer which may be micro-perforated to have the micro-perforations 402. In one example, the micro-perforations 402 may be formed post moulding of the continence membrane 118, by punching or piercing the continence membrane 118 with thin needles or any other perforation processes known in the art. The micro-perforations may allow gaseous materials to pass through while preventing any solid and liquid intestinal waste from passing through the micropores due to surface tension forces that prevent ingress of the non-gaseous materials into the pores. In one example, the perforation may have a hole size in the range of 10 microns to 50 microns.

Further, as illustrated in FIG. 4b, the intestinal waste management system 102 may include a cover or a cap 404 provided to fit over the front switch 112. In one example, the cap 404 may be detachably attached to the front switch 112 to enclose the continence membrane 118. The cap 404 may include a vent 406 to allow the gaseous waste matter to escape from the intestinal waste management system 102. Thus, the gases escaping from the micro-perforations 402 may pass through the cover or cap 404 and exit through the vent 406 which may allow the gas to escape in a controlled manner.

The cap 404 may further include an odour control unit 408 to absorb odour associated with flatus such that the gases pass through the odour control unit 408 before escaping into the surroundings, thus eliminating any odour associated with the flatus. In one example, the odour control unit 408 may be provided within the vent 406 and may contain an odour elimination substance, such as activated charcoal.

In another embodiment, the flatus release mechanism may include at least one multi-lumen discharge channel 410 embodied within an outer edge of the discharge channel 114 to allow the gaseous waste matter to escape from the intestine to outside the intestinal waste management system 102, as illustrated in FIG. 4c.

FIG. 4c illustrates a multi-lumen discharge channel 410 of the intestinal waste management system 102, in accordance with an embodiment of the present subject matter. The multi-lumen discharge channel 410 may function as the flatus management system be provided to allow the gas release. In one example, the multi-lumen discharge channel 410 may include one or more gas release lumens 412-1, 412-2, and 412-n, collectively referred to as gas release lumens 412 and individually as gas release lumen 412. The gas release lumens 412 may be formed within the multi-lumen discharge channel 410 to allow the gaseous waste matter to escape from the intestine to outside the intestinal waste management system 102. The gas release lumens 412 may have a small cross-sectional area—ID typically 0.2 mm-1 mm. Such small openings may selectively allow gaseous matter to continuously seep through and allow the gas to escape out of the stoma while preventing any solid and liquid intestinal waste to escape.

FIGS. 5a, 5b, and 5c illustrate the fluid valve 122 of the intestinal waste management system 102, in accordance with an embodiment of the present subject matter. FIG. 5a illustrates the fluid valve 122 of the intestinal waste management system 102. FIG. 5b illustrates a dome valve of the fluid valve 122 in a closed state, with the opening of the dome valve closed. FIG. 5c illustrates the dome valve in an open state, with the opening of the dome valve open.

As previously described, the fluid valve 122 may be connected to the inlet of the fixation element 120 for controlled inflation and deflation of the fixation element 120. In one example, the fluid valve 122 may include a valve top 502, a valve bottom 504, a valve body 506, and a dome valve 508. The dome valve 508 may be a non-return valve element, such as a duckbill type elastomeric dome valve. The dome valve 508 may be arranged to allow free flow of inflation medium from an inflation device, such as a syringe to the fixation element 120. Further, the dome valve 508 may prevent backflow of the inflation medium from the fixation element 120, thus preventing the fixation element 120 from deflating on its own.

In one example, the dome valve 508 may be seated inside the valve body 506 between the valve top 502 and the valve bottom 504. Upon assembly, the valve top 502 may create an inlet half of the fluid valve 122 and may have an over moulded elastomer membrane 510. The valve top 502 may also include a rigid actuator 512 coupled to the dome valve 508 to move the dome valve 508 to an open state from the closed state. In one example, upon pressing the over moulded elastomer membrane 510, the actuator 512 may move and open an opening of the dome valve 508. This action overrides the non-return functionality of the dome valve 508 to allow the backflow of the inflation medium from the fixation element 120 to the fluid valve 122, as illustrated in FIG. 5c.

The fluid valve 122 may further include an inlet 514 to receive the inflation medium from an inflation device to inflate the fixation element 120. In one example, the inflation device may be connected to the inlet 514 of the fluid valve 122 when the inflation medium is to be injected into the fixation element 120. The fluid valve 122 may further include an outlet 516 connected to the fixation element 120 through the inflation tubing 124. In one example, a first end of the inflation tubing 124 maybe connected to the outlet 516 and a second end may be connected to the fixation element 120 to provide a fluid path for the inflow and outflow of the inflation medium from the fixation element 120.

In one example, the outer body and the actuator 512 of the fluid valve 122 may be manufactured using Polypropylene or Polyethylene. The over moulded elastomer membrane 510 may be manufactured using thermoplastic elastomer, such as TPE, TPU, and Silicone Elastomer. Further, the dome valve 508 may be manufactured using material such as silicone rubber, TPE, and TPU. In one example, the fluid valve 122 may also include a mounting attachment 518.

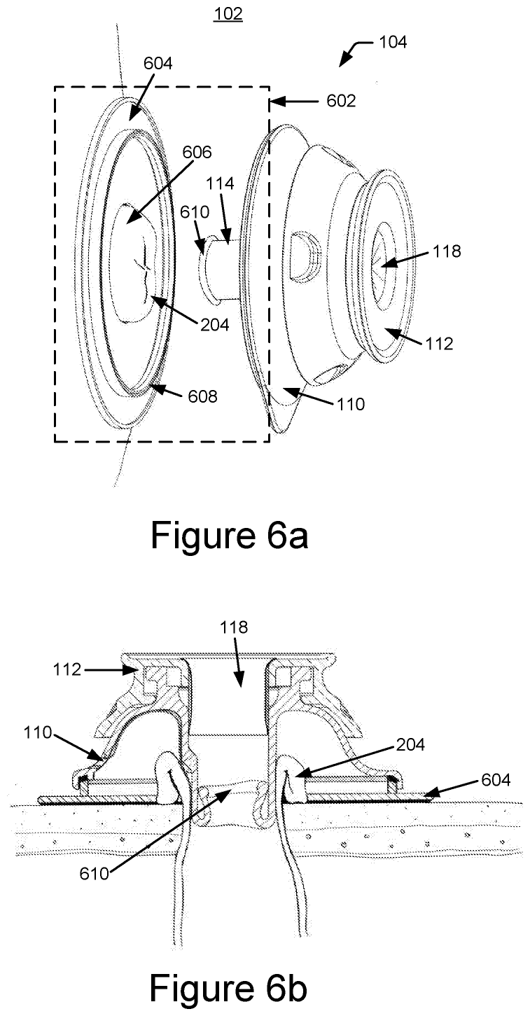

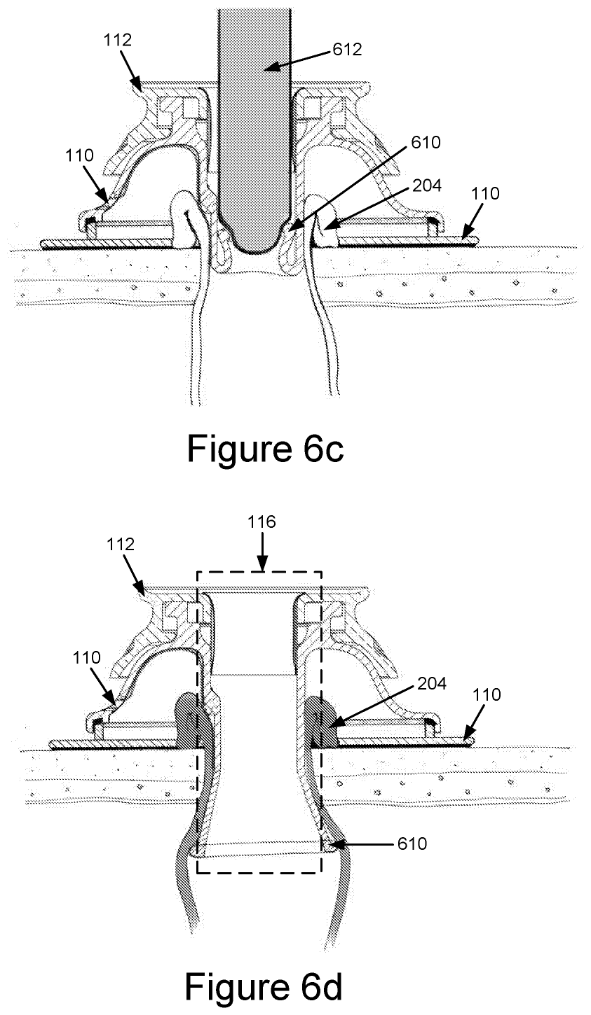

FIGS. 6a, 6b, 6c, and 6d illustrate the intestinal waste management system 102, in accordance with another embodiment of the present subject matter FIG. 6a illustrates an exploded view of the intestinal waste management system 102, in accordance with the other embodiment of the present subject matter. The intestinal waste management system 102 in accordance to the said embodiment may include a partially insertable device 104 having an anchoring mechanism 602 alternate to the anchoring mechanism 106. In said embodiment, the device 104 is partially insertable inside the stoma on the patient's abdomen. The intestinal waste management system 102, of the current embodiment, may be used by people having a stoma with sensitive intestinal lining and may thus be uncomfortable using an insertable anchoring mechanisms or may have comorbidities that are contraindicative for use of fixation elements like a balloon.

The anchoring mechanism 602 may include an adhesive backed plate 604. In one example, the adhesive backed plate 604 may be stuck onto the abdomen (peristomal area) and the discharge channel 114 of the device 104 may be introduced partially into the stoma through a slot 606 provided in the adhesive backed plate 604. The base flange 110 may then be snap fitted with a locking feature, such as a locking member 608 provide on the adhesive backed plate 604 to provide a leak proof connection. In one example, the proximal end of the discharge channel 114 may include a soft raised ring/collar 610 which may contact snugly with the mucosal lining to minimize leak and channelizes the intestinal effluents through the discharge channel 114.

In case of minor seepage of effluent around the ring or collar 610, the discharge may get collected into a dome shaped cavity within the base flange 110 and the adhesive backed plate 604, thereby preventing any contact of the effluent with the peristomal skin. The device 104 may further include an applicator 612 to unfold the collar 610 to create a continuous flow path with the discharge channel 114 for unrestricted flow of intestinal waste from within the intestine, to outside of the intestinal waste management system 102. As illustrated in FIG. 6c, once the device 104 is partially inserted inside the stoma, the applicator 612 may be inserted through the front switch 112 to unfold the collar 610 to create the continuous flow path with the discharge channel 114. The applicator 612 may be removed after the collar 610 is unfolded to create the continuous flow path with the discharge channel 114 as illustrated in the FIG. 6d.

The evacuation process and continence mechanism 108 of the device 104 may work in the manner as previously described with respect to other embodiments. For instance, the device 104 in said embodiment may include the continence mechanism 108 similar to the continence mechanism 108 as disclosed in previous Figures, to control discharge of intestinal waste from the intestine of the patient to outside the intestinal waste management system 102. The continence mechanism 108 may include the base flange 110 to be placed at the outer periphery of the stoma. As previously described, the base flange 110 maybe connected to the discharge channel 114 having the collar 610 at the proximal end to snugly contact with the mucosal lining of the stoma to create the discharge passage 116 to transport intestinal waste from the intestine to outside the intestinal waste management system 102, in the open state of the continence mechanism 108.

The continence mechanism 108 may further include the continence membrane 118 connected to the distal end of the discharge channel 114 such that in the closed state of the continence mechanism 108, the continence membrane may fold upon itself to create the constriction within the discharge channel 114 to stop the intestinal waste from flowing out from the intestinal waste management system 102. Further, the continence mechanism 108 may also include the rotation control mechanism 302 provided on the base flange 110 and the rotation control lug 304 protruding from the front switch 112, as previously described.

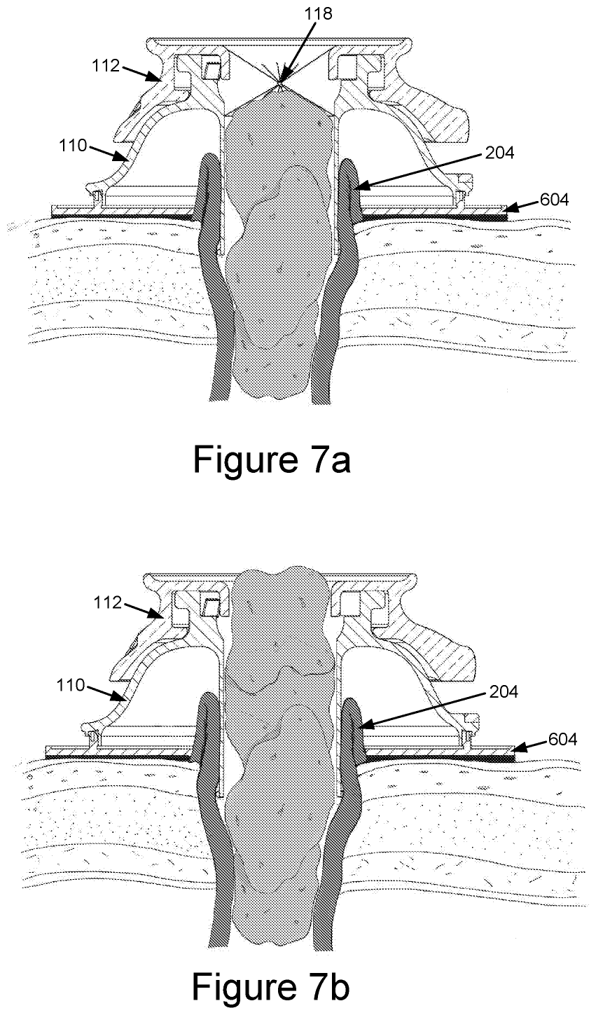

FIGS. 7a and 7b illustrate the intestinal waste management system 102 of FIGS. 6a-6c in different modes of operation, in accordance with another embodiment of the present subject matter. FIG. 7a illustrates the continence mechanism 108 with the continence membrane 118 in the closed state. FIG. 7b illustrates the continence mechanism 108 with the continence membrane in the open state, allowing the stool to exit the device 104. As previously described, the evacuation process and continence mechanism 108 of the device 104 of FIGS. 6a-6d may work in the manner as previously described in FIGS. 1-5 with respect to other embodiments.

For instance, to use the intestinal waste management system 102, the adhesive backed plate 604 may be initially stuck onto the abdomen of the user. The discharge channel 114 of the device 104 may then be partially inserted into the stoma through the slot 606. The base flange 110 may then be snap fitted on the adhesive backed plate 604 and the continence membrane 118 may be closed using the front switch 112. Once the device 104 is anchored, the user may continue with their daily activities with the continence mechanism 108 in the closed state as illustrated in FIG. 7a. As previously described, in the closed state, the continence membrane 118 may fold upon itself to create a constriction within the discharge passage 116, thereby, closing the flow path for the waste matter within the discharge passage 116. The constriction created due to the continence membrane 118 prevents the intestinal waste from exiting the device 104.

To empty the contents of the intestine, the user may attach the waste collection bag 128 to the front switch 112 and rotate the front switch 112 to move the continence mechanism 108 into the open state. The intestinal waste collected in the discharge passage 116 may then ooze out, as illustrated in the FIG. 7b.

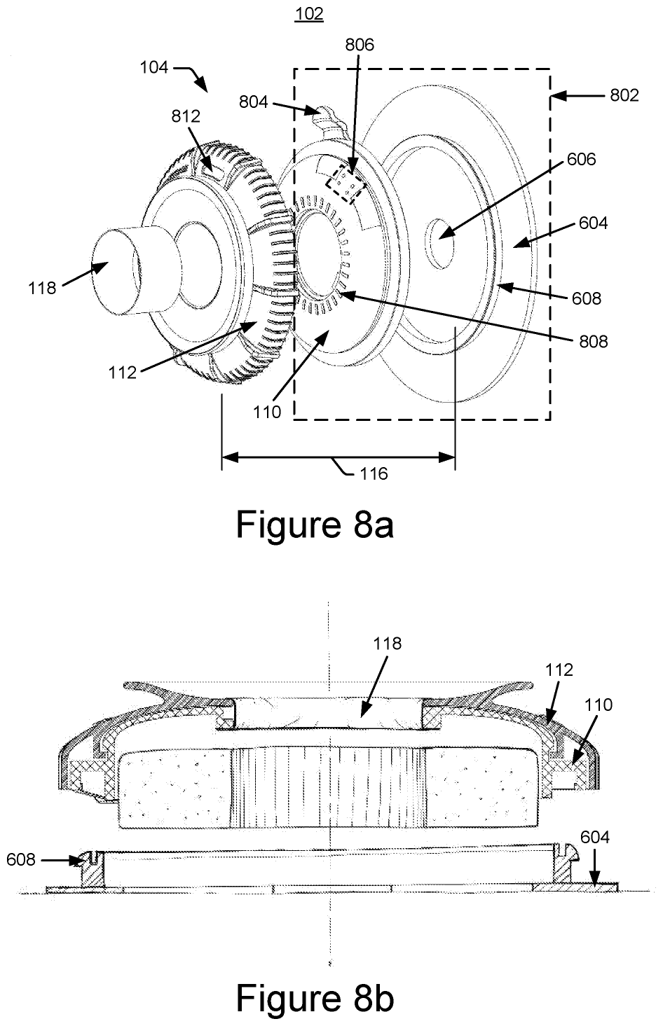

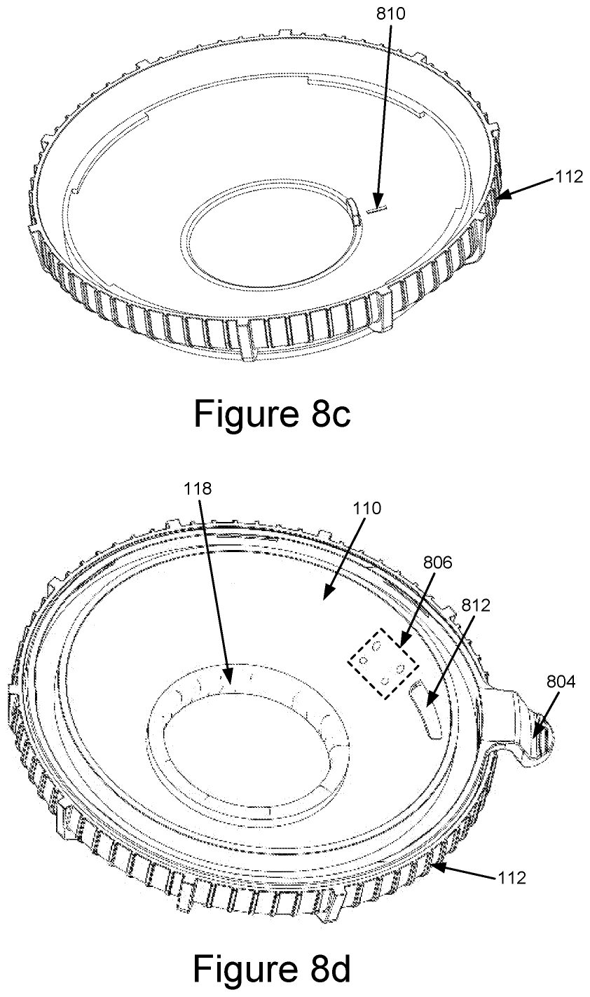

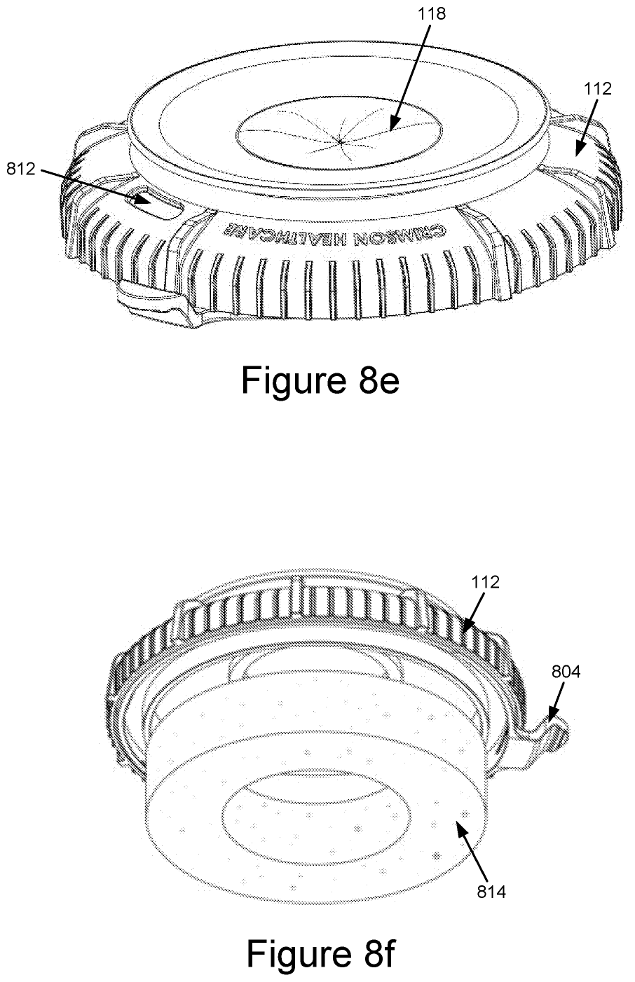

FIGS. 8a, 8b, 8c, and 8d illustrate the intestinal waste management system 102, in accordance with yet another embodiment of the present subject matter. FIG. 8a illustrates an exploded view of the intestinal waste management system 102, in accordance with yet another embodiment of the present subject matter. FIG. 8b illustrates a cross section of the base flange 110 and the front switch 112. FIG. 8c illustrates a bottom view of the front switch 112. FIG. 8d illustrates a bottom view of the base flange 110 snap fitted onto the front switch 112. FIG. 8e illustrates a top view of the base flange 110 snap fitted onto the front switch 112. FIG. 8f illustrates the base flange 110 and an odour control component of the intestinal waste management system 102.

The intestinal waste management system 102 in accordance to the said embodiment includes a non-insertable device 104 having an anchoring mechanism 802 alternate to the anchoring mechanism 106. In said embodiment, the device 104 is non insertable and may thus be used by people having a stoma with sensitive intestinal lining and may thus be uncomfortable using insertable or partially insertable anchoring mechanisms.

As illustrated in FIG. 8a, the anchoring mechanism 802 may include the adhesive backed plate 604, the slot 606, and the locking member 608 similar to the anchoring mechanism 602. As previously explained, the adhesive backed plate 604 may be stuck onto the abdomen (peristomal area) to anchor the device 104 of the intestinal waste management system 102.

The locking member 608 may be provided on the adhesive backed plate 604 to receive the base flange 110 such the base flange 110 may be snap fitted with the locking member 608 to provide a leak proof connection.

In one example, the device 104 may include the continence mechanism 108 to control discharge of intestinal waste from the intestine of the patient to outside the intestinal waste management system 102, as described with reference to FIGS. 1-7. In one example, the continence mechanism 108 may include the base flange 110 to be placed at an outer periphery of the stoma to form the discharge passage 116 to transport intestinal waste from the intestine to outside the intestinal waste management system 102, in an open state of the continence mechanism 108. The continence mechanism 108 may further include the continence membrane 118 having the first end connected to the base flange 110 to form the discharge passage 1176. The continence membrane may fold upon itself to create a constriction within the discharge passage 116 to stop the intestinal waste from flowing out from the intestinal waste management system 102. The continence mechanism 108 may further include the front switch 112, detachably connected to the base flange 110, as illustrated un FIGS. 8d and 8e. As illustrated, a proximate end of the front switch 112 maybe connected to a second end of the continence membrane 118 to control an operational state of the continence mechanism 108 between the open state and the close state. The evacuation process and continence mechanism 108 of the device 104 may work in the manner as previously described with respect to other embodiments. For instance, the continence mechanism 108 may also include the rotation control mechanism 302 provided on the base flange 110 and the rotation control lug 304 protruding from the front switch 112, as previously described.

In one example, the base flange 110 may include one or more detachment tab(s) 804 to detach the device 104 from the adhesive backed plate 604. In one example, the detachment tab 804 may also serve as an orientational cue at the time of application while snapping the device 104 onto the adhesive backed plate 604 to ensure that the device 104 is mounted in an appropriate orientation. The base flange 110 may further include a plurality of pores 806 to allow gaseous waste matter to passively escape in a closed state of the continence mechanism 108. The plurality of pores 806 may allow controlled discharge of the gaseous waste matter from the intestine to outside the intestinal waste management system 102. The base flange 110 may further include a plurality of raised features 808 to prevent the front switch 112 from unintended rolling back to prevent any unwanted opening of the continence membrane 118. In one example, the front switch 112 may include one or more detents 810 on an interior surface, as illustrated in FIG. 8c, to engage with the raised features 808 of the base flange 110 to prevent accidental rotation of the front switch 112 in an assembled state of the device 104.

The front switch 112 may further include a state indicator 812 to indicate an operational state of the continence membrane 118. In one example, the state indicator 812 may include a window and a visual indicator to indicate the open and closed state of the continence membrane 118. In one example, the visual indicator may be color coded, say, green color to indicate closed state and red color to indicate open state. In another example, the visual indicator may include text, such as “closed” and “open”. In another example, the visual indicator may include other means of visual indication.

In one example, the device 104 may further include an odour control component 814. As illustrated in FIG. 8f, the odour control component 814 may be a ring-shaped component manufactured using a porous and flexible substance, such as impregnated foam infused with an odour elimination substance. In one example, the odour elimination substance, maybe activated charcoal.

As illustrated in FIG. 8f, the odour control component 814 may be housed inside a cavity of the base flange 110, such that the odour of gaseous waste matter released through the stomal opening is neutralized before being released through the pores 806 of the base flange 110. Further, the odour control component 814 may be snugly fitted inside the base flange 110 to constitute the discharge passage 116 for evacuation of the intestinal waste. In one example, the odour control component 814 may occupy a large volume of the cavity of the base flange 110, thereby, leaving little space for intestinal waste to get accumulated. The odour control component 814 may thus facilitate in preventing the intestinal waste from creeping into a narrow gap between the slot 606 of the adhesive backed plate 604 and the stoma. The odour control component 814 may thus prevent liquid/solid from passing through, while allowing gases to escape due to its porous construction.

Although examples for the present subject matter have been described in language specific to structural features and/or methods, it should be understood that the appended claims are not limited to the specific features or methods described. Rather, the specific features and methods are disclosed and explained as examples of the present subject matter.

1. An intestinal waste management system comprising:
a device, insertable inside a stoma on a patient's abdomen, the device comprising:
an anchoring mechanism for anchoring the device once the device is inserted into the stoma of a patient; and
a continence mechanism to control discharge of intestinal waste from an intestine of the patient to outside the intestinal waste management system, the continence mechanism comprising:
a base flange to be placed at an outer periphery of the stoma, wherein the base flange is connected to a discharge channel to form a discharge passage to transport intestinal waste from the intestine to outside the intestinal waste management system, in an open state of the continence mechanism, wherein the discharge passage is formed between a proximal end of the base flange and a distal end of the anchoring mechanism; and
a continence membrane having a first end connected to a distal end of the discharge channel, wherein in a closed state of the continence mechanism, the continence membrane is to fold upon itself to create a constriction within the discharge passage to stop the intestinal waste from flowing out from the intestinal waste management system.

2. (canceled)

3. The intestinal waste management system as claimed in claim 1, wherein the continence membrane is to unfold in the open state of the continence mechanism to create a continuous flow path with the discharge passage for unrestricted flow of waste matter from within the intestine to outside of the intestinal waste management system.

4. The intestinal waste management system as claimed in claim 1, wherein the continence mechanism 108 further includes a front switch disposed on the base flange, wherein a proximate end of the front switch is connected to a second end of the continence membrane to control an operational state of the continence mechanism between the open state and the closed state.

5. The intestinal waste management system as claimed in claim 4, wherein the continence mechanism comprises:
a rotation control mechanism provided on the base flange; and
a rotation control lug protruding from the front switch, wherein the rotation control lug is to engage with the rotation control mechanism to control a rotational movement of the front switch along an outer surface of the base flange.

6. The intestinal waste management system as claimed in claim 5, wherein the rotation control mechanism comprises an end stop to engage with the rotation control lug to stop the front switch from rotating in a first direction in the closed state of the continence mechanism and in a second direction in the open state of the continence mechanism.

7. The intestinal waste management system as claimed in claim 6, wherein the rotation control mechanism further comprises a control step provided on the base flange to provide resistance to movement of the rotation control lug to prevent any unwanted opening of the continence membrane.

8. The intestinal waste management system as claimed in claim 6, wherein the base flange comprises a first step and a second step to provide resistance to movement of the rotation control lug to provide a haptic feedback to a user operating the intestinal waste management system, as an indication of the continence mechanism entering the closed state.

9. The intestinal waste management system as claimed in claim 5, further comprising a switch housing disposed on the base flange and the front switch to restrain an axial movement of the front switch, wherein the front switch is rotatable about a central axis.

10. The intestinal waste management system as claimed in claim 9, wherein the switch housing comprises at least one cut-out window on an outer circumference to allow physical access to a hand grip to operate the front switch.

11. The intestinal waste management system as claimed in claim 1, wherein the intestinal waste management system comprises a flatus release mechanism to allow controlled discharge of gaseous waste matter from the intestine to outside the intestinal waste management system.

12. (canceled)

13. The intestinal waste management system as claimed in claim 11, wherein the flatus release mechanism comprises:
a first step provided on the base flange to receive a rotation control lug of the continence mechanism for partially opening the continence membrane to a first partial position to allow the gaseous waste matter to escape from the intestine to outside the intestinal waste management system; and
a second step provided on the base flange to receive the rotation control lug for partially opening the continence membrane up to a second partial position to allow the gaseous waste matter to escape from the intestine to outside the intestinal waste management system.

14. (canceled)

15. The intestinal waste management system, as claimed in claim 11, wherein the flatus release mechanism comprises at least one multi-lumen discharge channel embodied within an outer edge of the discharge channel to allow the gaseous waste matter to escape from the intestine to outside the intestinal waste management system.

16. The intestinal waste management system as claimed in claim 1, wherein the anchoring mechanism comprises:
a fixation element disposed at a proximal end of the device, to anchor the intestinal waste management system inside the intestine once the device is inserted into the intestine, wherein the fixation element is to inflate to anchor the intestinal waste management system inside the intestine, and wherein the fixation element is to deflate to allow removal of the intestinal waste management system from the intestine;
a fluid valve to regulate inflow and outflow of an inflation medium from the fixation element to control inflation and deflation of the fixation element, wherein an inlet of the fluid valve is to receive the inflation medium from an inflation device to inflate the fixation element; and
an inflation tubing having a first end connected to an outlet of the fluid valve and a second end connected to the fixation element to provide a fluid path for the inflow and outflow of the inflation medium from the fixation element.

17. (canceled)

18. The intestinal waste management system as claimed in claim 1, wherein the intestinal waste management system comprises a waste collection bag, connectable to an outer ring of a front switch of the device, to collect the intestinal waste flowing out of the front switch through the continence membrane and the discharge passage.

19. The intestinal waste management system as claimed in claim 1, further comprising an intestinal pressure measurement and monitoring unit comprising:
a sensor to measure intestinal pressure developing with the intestine of the patient;
a communication module to:
communicate pressure information indicating the intestinal pressure to a user device; and
send an alarm to the user device when the intestinal pressure exceeds a predetermined threshold level; and
an inflation pressure monitor to monitor if the user has filled a fixation element of the device up to a predetermined level while also limiting the user from over-inflation.

20. An intestinal waste management system comprising:
a device, partially insertable inside a stoma on a patient's abdomen, the device comprising:
a continence mechanism to control discharge of intestinal waste from an intestine of the patient to outside the intestinal waste management system, the continence mechanism comprising:
a base flange to be placed at an outer periphery of the stoma, wherein the base flange is connected to a discharge channel having a collar at a proximal end to snugly contact with a mucosal lining of the stoma to create a discharge passage to transport intestinal waste from the intestine to outside the intestinal waste management system, in an open state of the continence mechanism; and
an anchoring mechanism for anchoring the device on abdomen of the patient, wherein the anchoring mechanism comprises:
an adhesive backed plate to be stuck onto the abdomen of the patient; and
a locking member provided on the adhesive backed plate to receive the base flange, wherein the base flange is to snap fit with the locking member.

21. The intestinal waste management system as claimed in claim 20, wherein the discharge channel is to be partially inserted into the stoma through a slot provided in the adhesive backed plate.

22. The intestinal waste management system as claimed in claim 21, further comprising an applicator to unfold the collar to create a continuous flow path with the discharge channel for unrestricted flow of the intestinal waste from within the intestine, to outside of the intestinal waste management system.

23. The intestinal waste management system as claimed in claim 20, wherein the continence mechanism comprises a continence membrane connected to a distal end of the discharge channel, wherein in a closed state of the continence mechanism, the continence membrane is to fold upon itself to create a constriction within the discharge channel to stop the intestinal waste from flowing out from the intestinal waste management system, and
wherein the continence membrane is to unfold in the open state of the continence mechanism to create a continuous flow path with the discharge passage for unrestricted flow of waste matter from within the intestine to outside of the intestinal waste management system.

24. (canceled)

25. The intestinal waste management system as claimed in claim 23, wherein the continence mechanism further includes a front switch disposed on the base flange, wherein a proximate end of the front switch is connected to a second end of the continence membrane to control an operational state of the continence mechanism between the open state and the closed state.

26.-30. (canceled)

31. An intestinal waste management system comprising:
a device, attachable to a stoma on a patient's abdomen, the device comprising:
a continence mechanism to control discharge of intestinal waste from an intestine of the patient to outside the intestinal waste management system, the continence mechanism comprising:
a base flange to be placed at an outer periphery of the stoma, wherein the base flange is to form a discharge passage to transport intestinal waste from the intestine to outside the intestinal waste management system, in an open state of the continence mechanism; and
an anchoring mechanism for anchoring the device on the abdomen of the patient, wherein the anchoring mechanism comprises:
an adhesive backed plate to be stuck onto the abdomen of the patient; and
a locking member provided on the adhesive backed plate to receive the base flange, wherein the base flange is to snap fit with the locking member.

32. The intestinal waste management system as claimed in claim 31, wherein the continence mechanism comprises a continence membrane having a first end connected to the base flange to form the discharge passage, wherein in a closed state of the continence mechanism, the continence membrane is to fold upon itself to create a constriction within the discharge passage to stop the intestinal waste from flowing out from the intestinal waste management system; and
wherein the continence membrane is to unfold in an open state of the continence mechanism to create a continuous flow path with the discharge passage for unrestricted flow of waste matter from within the intestine to outside of the intestinal waste management system.

33. (canceled)

34. The intestinal waste management system as claimed in claim 32, wherein the continence mechanism further includes a front switch disposed on the base flange, wherein a proximate end of the front switch is connected to a second end of the continence membrane to control an operational state of the continence mechanism between the open state and the closed state.

35.-38. (canceled)

39. The intestinal waste management system as claimed in claim 34, wherein:
the base flange includes a plurality of raised features; and
the front switch includes one or more detents on an interior surface of the front switch to engage with the plurality of raised features of the base flange to prevent accidental rotation of the front switch in an assembled state of the device.

40. The intestinal waste management system as claimed in claim 31, wherein the base flange comprises one or more detachment tabs to detach the device from the adhesive backed plate.

41. The intestinal waste management system as claimed in claim 31, wherein the base flange comprises:
a plurality of pores to allow controlled discharge of gaseous waste matter from the intestine to outside the intestinal waste management system; and
an odour control component housed inside a cavity of the base flange to neutralize odour of the gaseous waste matter before being released through the pores, wherein the odour control component is a porous substance infused with an odour elimination substance to neutralize odour of the gaseous waste matter.
